(19) United States
(12) Patent Application Publication
(10) Pub. No.: US 2007/0197467 A1
(43) Pub. Date: Aug. 23, 2007

(54) ZAFIRLUKAST COMPOSITIONS

(76) Inventors: Srinivasulu Rangineni, Mahabubnagar 509 102, Andhra Pradesh (IN); Harshal Prabhakar Bhagwatwar, Pune 411 029, Maharashtra (IN); Surya Narayana Devarakonda, Hyderabad 500 047, Andhra Pradesh (IN); Sudeep Kumar Agarwal, Raipur 492 010, Chhattisgarh (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/671,480

(22) Filed: Feb. 6, 2007

Related U.S. Application Data
(60) Provisional application No. 60/804,835, filed on Jun. 15, 2006.

(30) Foreign Application Priority Data
Feb. 6, 2006 (IN) 182/CHE/2006

(51) Int. Cl. A61K 31/47; A61K 31/717; C07D 209/56

(52) U.S. Cl. 514/57; 514/410; 536/56; 548/418; 514/311

(57) ABSTRACT

Intimate dispersions comprising zafirlukast and hydroxypropyl cellulose.

### INTRODUCTION TO THE INVENTION

The present invention relates zafirlukast compositions. More particularly it relates to stable compositions comprising zafirlukast and hydroxypropyl cellulose (HPC), and a process for their preparation.

Zafirlukast has the chemical name 4-(5-cyclopentyloxy-carbonylamino-1-methyl-indol-3-ylmethyl)-3-methoxy-N-o-tolylsulfonylbenzamide and is structurally represented by Formula 1.

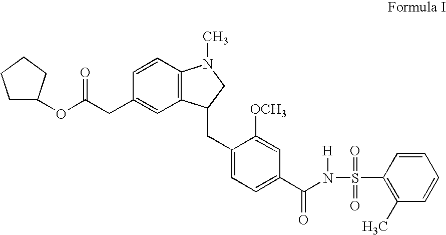

Zafirlukast is a synthetic, selective peptide leukotriene receptor antagonist (LTRA), useful for the treatment of asthma and is commercially available in the market under the brand name ACCOLATE™ supplied as 10 and 20 mg tablets for oral administration.

U.S. Pat. No. 5,391,097 discloses zafirlukast, its pharmaceutical compositions, and processes for their preparation.

U.S. Pat. No. 5,482,963 discloses a pharmaceutical composition which comprises amorphous zafirlukast and polyvinylpyrrolidone (PVP).

U.S. Pat. No. 5,612,367 discloses a method for stabilizing amorphous Form A of zafirlukast using PVP.

U.S. Pat. No. 5,993,859 discloses a process for the preparation of zafirlukast coprecipitate with PVP by mixing zafirlukast, PVP, and water and drying the mixture.

U.S. Pat. No. 6,143,775 discloses amorphous Form A of zafirlukast substantially free of other physical forms.

Other crystalline forms of zafirlukast and their compositions have been disclosed in U.S. Pat. Nos. 5,319,097 and 5,294,636.

It is sometimes desired to form a composition of a drug and a polymer. One reason for forming such compositions is that the aqueous concentration of a poorly soluble drug may be improved by such a technique. For example, European Patent Application No. 0 901 786 A2 discloses forming pharmaceutical spray-dried dispersions of sparingly soluble drugs and the polymer hydroxypropyl methylcellulose, in which the drug is amorphous and dispersed in the polymer. The spray-dried dispersions disclosed in the patent provide superior aqueous concentration relative to dispersions formed from other methods and relative to the crystalline drug alone.

None of the patents described above have described a stable composition of zafirlukast with HPC.

The bioavailability of an orally administered drug, as measured by its entry into systemic circulation in the bloodstream, depends on at least two fundamental processes: drug dissolution in gastrointestinal fluids (in vivo drug release) and subsequent absorption of the dissolved drug. Several factors influence dissolution of a drug from its carrier, including surface area of the drug presented to the dissolution solvent medium, solubility of the drug substance in the solvent medium, and driving forces of the saturation concentration of dissolved materials in the solvent medium.

Towards this end, it has been the endeavor of pharmaceutical scientists to provide a new crystalline form of zafirlukast, more specifically, a thermodynamically stable form which would have the strengths of the crystalline forms, viz. thermodynamic stability, and those of the amorphous form, viz. enhanced solubility, rapid onset of action and an enhanced bioavailability.

Consequently, there is a need for soluble forms of zafirlukast that can be readily formulated for use in various modes of administration, including parenteral and oral administration.

The present invention provides compositions comprising zafirlukast and hydroxypropylcellulose (HPC), which are stable and are reproducibly prepared on an industrial scale.

### SUMMARY OF THE INVENTION

The present invention relates to stable compositions comprising zafirlukast and HPC, and processes for their preparation.

One aspect of the present invention provides a stable composition of zafirlukast with HPC, characterized by its X-ray powder diffraction (“XRPD”) pattern, and differential scanning calorimetry (“DSC”) curve.

Another aspect of the present invention provides a process for the preparation of stable composition of zafirlukast with HPC. In an embodiment, a process for the preparation of a stable composition comprising zafirlukast and HPC comprises:

a) providing a solution comprising zafirlukast and HPC in a suitable solvent; and

b) removing solvent from the solution of a).

Still another aspect of the present invention provides a pharmaceutical composition comprising a stable composition of zafirlukast with HPC, in combination with one or more pharmaceutically acceptable excipients.

### BRIEF DESCRIPTION OF THE DRAWINGS

FIG. 1 is an X-ray powder diffraction pattern of a stable composition comprising zafirlukast and HPC, prepared in Example 1.

FIG. 2 is a differential scanning calorimetric curve of a stable composition comprising zafirlukast and HPC, prepared in Example 1.

### DETAILED DESCRIPTION OF THE INVENTION

The present invention relates to stable compositions comprising zafirlukast and HPC, and processes for their preparation.

One aspect of the present invention provides stable compositions comprising zafirlukast and HPC characterized by their X-ray powder diffraction (“XRPD”) patterns, and differential scanning calorimetry (“DSC”) curves.

A stable composition comprising zafirlukast and HPC of the present invention may contain at least a detectable amount of zafirlukast present therein in amorphous form. It is believed that amorphous zafirlukast particles require less energy for dissolution than crystalline zafirlukast particles of similar dimensions, and that this reduced dissolution energy requirement contributes, at least in part, to an increased dissolution rate and decreased therapeutic onset time exhibited by amorphous zafirlukast and compositions thereof. In embodiments of the invention, the zafirlukast in the stable composition is substantailly amorphous, meaning that less than about 5 percent by weight of the contained zafirlukast is in a crystalline form; crystallinity can be detected and quantified by known methods such as X-ray powder diffraction.

In embodiments, about 10% to 100%, or about 25% to 100%, or about 60% to 100%, by weight, of zafirlukast present in the composition is in the amorphous form.

All XRPD data reported herein were obtained using Cu Kα-1 radiation, having the wavelength 1.541Å, and were generated using a Bruker Axe, D8 Advance Powder X-ray Diffractometer.

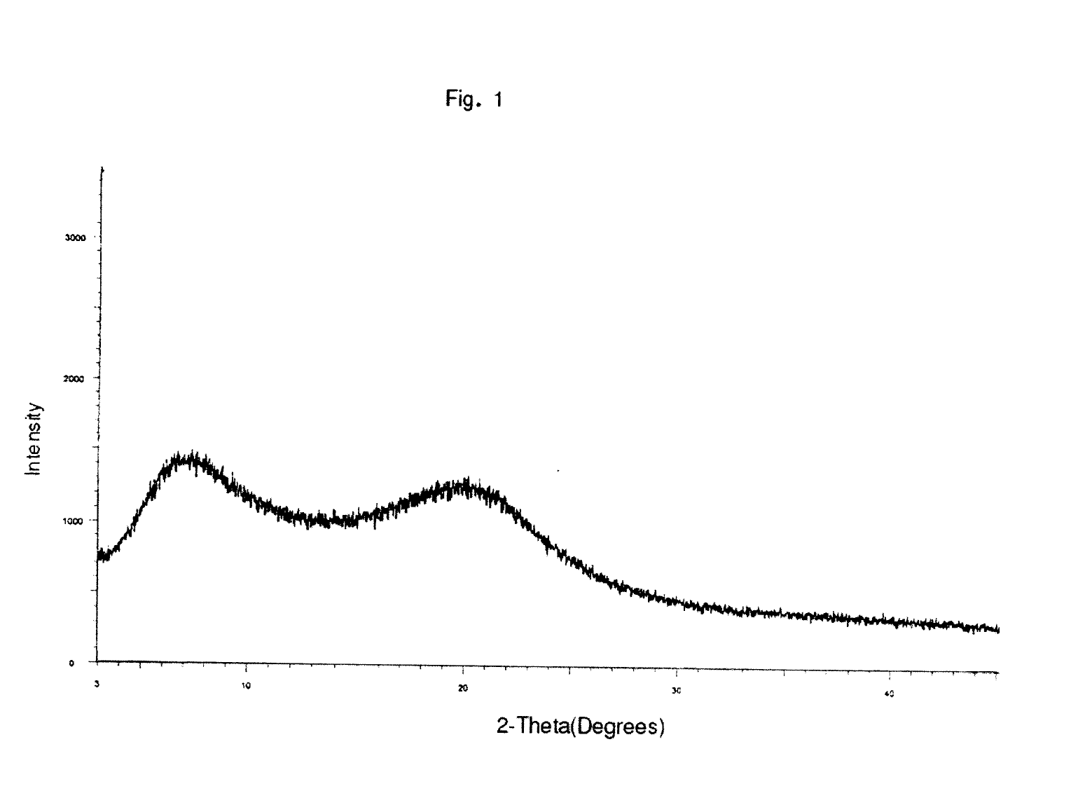

In certain embodiments, stable compositions comprising amorphous zafirlukast and HPC can be characterized by their XRPD patterns showing a plain halo with no peaks, which is characteristic of an amorphous solid, substantially in accordance with FIG. 1.

Differential scanning calorimetric analysis was carried out using a DSC Q1000 model from TA Instruments with a ramp of 5° C./minute. The starting temperature was 40° C. and ending temperature was 200° C.

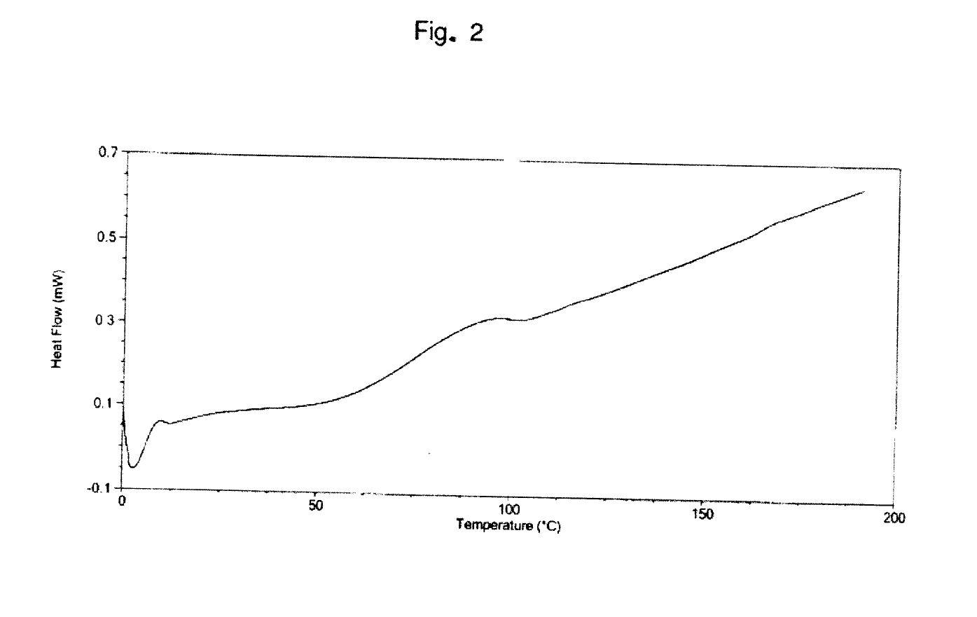

In an embodiment, a stable composition comprising zafirlukast and HPC has a characteristic differential scanning calorimetry curve substantially in accordance with FIG. 2, having an onset of glass transition at about 94° C., a half point glass transition at about 101° C. and ending of glass transition at about 108° C.

Another aspect of the present invention provides a process for the preparation of a stable composition comprising zafirlukast and HPC. In an embodiment, the process comprises:

a) providing a solution comprising zafirlukast and HPC in a suitable solvent;

b) removing solvent from the solution of a).

Step a) involves providing a solution comprising zafirlukast and HPC in a suitable solvent.

The solution of zafirlukast and HPC may be obtained by dissolving zafirlukast and HPC in a suitable solvent, or such a solution may be obtained directly from a reaction in which zafirlukast is formed, and HPC subsequently added to it.

When the solution is prepared by dissolving zafirlukast in a solvent, any form of zafirlukast such as an amorphous or any crystalline form of zafirlukast, including any solvates and hydrates, may be utilized for preparing the solution. In an embodiment, separate solutions of zafirlukast and HPC are prepared, then the solutions are combined before step b) is conducted.

HPC is a partially substituted poly(hydroxypropyl) ether of cellulose. HPC is commercially available in a number of different grades, which have different solution viscosities. The molecular weight of the HPC ranges from about 50,000 to about 1,250,000. A useful HPC is available from Aqualon (a unit of Hercules Industrial Chemicals Pvt. Ltd., Mumbai, India) under the trademark KLUCEL. Suitable grades of HPC include: KLUCEL EF having a molecular weight of about 80,000; KLUCEL LF having a molecular weight of about 95,000; KLUCEL JF having a molecular weight of about 140,000; KLUCEL GF having a molecular weight of about 370,000; KLUCEL MF having a molecular weight of about 850,000; and KLUCEL HF having a molecular weight of about 1,150,000. There are other suppliers of suitable HPC products, including Spectrum Chemical and Laboratory Products, California USA, Ruger Chemical Co. Ltd., New Jersey USA, and Biddle Sawyer Corporation, New York, USA.

When the solution comprising zafirlukast and HPC is prepared, the order of charging the two ingredients into the solvent is not critical for the product obtained. A specific order may be preferred with respect to the equipment actually used and will be easily determined by a person skilled in the art. In any case, zafirlukast must be completely soluble in the solvent of the invention and should provide a clear solution.

Suitably, both zafirlukast and HPC are added together to the solvent and dissolved to obtain a clear solution in the solvent.

The ratio of HPC to zafirlukast can range from about 0.1:99.9 to about 1:1 w/w.

HPC can be added directly, or it can be dissolved in a solvent before addition to the solution of zafirlukast.

Suitable solvents which can be used for dissolving zafirlukast either alone or along with HPC include but are not limited to: alcohols such as methanol, ethanol, isopropyl alcohol, n-propanol, and the like; halogenated hydrocarbons such as dichloromethane, 1,2-dichloroethane, chloroform, carbon tetrachloride and the like; ketones such as acetone, ethyl methyl ketone, methyl isobutyl ketone and the like; esters such as ethyl acetate, n-propyl acetate, n-butyl acetate, t-butyl acetate and the like; ethers such as diethyl ether, dimethyl ether, diisopropyl ether, 1,4-dioxane and the like; hydrocarbons such as toluene, xylene, n-heptane, cyclohexane, n-hexane and the like; nitriles such as acetonitrile, propionitrile and the like; and mixtures thereof or their combinations with water in various proportions.

The dissolution temperatures can range from about 20 to 120° C. depending on the solvent used for dissolution. Any other temperature is also acceptable as long as a clear solution is obtained.

The quantity of solvent used for dissolution depends on the solvent and the dissolution temperature adopted. The concentration of zafirlukast in the solution may generally range from about 0.1 to about 10 g/ml.

These lists of solvents are merely representative of those that can be used, and the lists are not intended to be exhaustive.

The solution may optionally be treated with materials such as carbon or sodium sulfate for clarification.

Optionally, the solution obtained above can be filtered to remove any undissolved particles followed by further processing. The undissolved particles can be removed suitably by filtration, centrifugation, decantation, and other techniques. The solution can be filtered by passing through paper, glass fiber, or other membrane material, or a bed of a clarifying agent such as celite. Depending upon the equipment used and the concentration and temperature of the solution, the filtration apparatus may need to be preheated to avoid premature crystallization.

Step b) involves removal of solvent from the solution of a).

Removal of the solvent may be carried out suitably using evaporation, atmospheric distillation, or distillation under vacuum.

Distillation of the solvent may be conducted under a vacuum, such as below about 100 mm Hg to below about 600 mm Hg, and/or at elevated temperatures such as about 20° C. to about 70° C. Any temperature and vacuum conditions can be used as long as there is no increase in the impurity levels of the product.

Suitable techniques which can be used for the distillation include, distillation using a rotational evaporator device such as a Buchi Rotavapor, spray drying, agitated thin film drying (“ATFD”), and the like.

These techniques are applicable to both aqueous and organic solutions of zafirlukast and mixtures of zafirlukast with a pharmaceutically acceptable carrier. However, solutions using the more volatile organic solvents are preferred.

Techniques such as Buchi Rotavapor drying and dry distillation under vacuum, may be suitable for laboratory-scale processes such as for quantities less than about 100 g. Other techniques such as spray drying and ATFD are more suitable for industrial scale production with a batch size of at least about 100 g or about 1 kg, or greater.

The zafirlukast composition obtained from step b) can be collected from the equipment using techniques such as by scraping, or by shaking the container, or other techniques appropriate for the particular apparatus being used. The composition will be in the form of an intimate dispersion comprising zafirlukast and hydroxypropyl cellulose, wherein specific regions of an individual component cannot be identified. The intimate dispersion is considered to be in the nature of a solid solution.

Optionally, the obtained product is further dried. Drying can be carried out at reduced pressures, such as below about 200 mm Hg or below about 50 mm Hg, at temperatures such as about 35° C. to about 70° C. Drying may be carried out for variable periods of time depending on the desired product purity to be obtained.

Drying can be suitably carried out in a tray dryer, vacuum oven, air oven, or using a fluidized bed drier, spin flash dryer, flash dryer and the like.

Drying can be carried out under reduced pressure until the residual solvent content reduces to an amount that is within the limits given by the International Conference on Harmonization of Technical Requirements for Registration of Pharmaceuticals for Human Use (“ICH”) guidelines. The guideline solvent level depends on the type of solvent but is not more than about 5000 ppm, or about 4000 ppm, or about 3000 ppm.

The dried product can optionally be milled to get a desired particle size distribution. Milling or micronization can be performed prior to drying, or after the completion of drying of the product. The milling operation reduces the size of particles and increases surface area of particles by colliding particles with each other at high velocities.

Drying is more efficient when the particle size of the material is smaller and the surface area is higher, hence milling can be performed prior to the drying operation.

Milling can be done suitably using jet milling equipment like an air jet mill, or using other conventional milling equipment.

The process of the present invention provides stable compositions of zafirlukast with HPC. The term “stable composition of zafirlukast” refers to stability of the polymorphic form under the standard temperature and humidity conditions of testing of pharmaceutical products, wherein the stability is evaluated by preservation of the original polymorphic form.

Stable compositions of zafirlukast with HPC obtained in this invention contain less than about 5000 ppm, or less than about 3000 ppm, or less than about 1000 ppm of methanol, and less than about 200 ppm, or less than about 100 ppm of other individual residual organic solvents.

In an embodiment, zafirlukast compositions with HPC prepared according to a process of the present invention comprise from about 10% to about 80%, or from about 15% to about 75%, or from about 25% to about 65%, by weight, of HPC.

Still another aspect of the present invention provides a pharmaceutical composition comprising a stable composition of zafirlukast with HPC and one or more pharmaceutically acceptable excipients.

The pharmaceutical composition comprising a stable composition of zafirlukast with HPC of the present invention may further formulated as: solid oral dosage forms such as, but not limited to, powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the form of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared by direct blending, dry granulation or wet granulation or by extrusion and spheronization. Compositions may be presented as uncoated, film coated, sugar coated, powder coated, enteric coated or modified release coated. Compositions of the present invention may further comprise one or more pharmaceutically acceptable excipients.

Pharmaceutically acceptable excipients that find use in the present invention include, but are not limited to: diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinyl pyrrolidone, hydroxypropyl cellulose, hydroxypropyl methylcellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins, resins; release rate controlling agents such as hydroxypropyl cellulose, hydroxymethyl cellulose, hydroxypropyl methylcellulose, ethyl cellulose, methyl cellulose, various grades of methyl methacrylates, waxes and the like. Other pharmaceutically acceptable excipients that are of use include but are not limited to film formers, plasticizers, colorants, flavoring agents, sweeteners, viscosity enhancers, preservatives, antioxidants and the like.

In the compositions of present invention zafirlukast is a useful active ingredient in the range of 5 mg to 10 mg, or 15 mg to 20 mg, and 30 mg, per dosage unit.

Certain specific aspects and embodiments of this invention are described in further detail by the examples below, which examples are provided only for the purpose of illustration and are not intended to limit the scope of the appended claims in any manner.

### EXAMPLE 1

Preparation of Stable Composition of Zafiflukast and Hydroxypropyl Cellulose

3 g of zafirlukast, 3 g of HPC (Klucel LF, mol. wt. 95000) and 200 ml of dichloromethane were taken into a clean and dry round bottom flask and stirred for 30 minutes at 25° C. The resultant reaction solution was passed through a filter paper to obtain a particle-free clear solution. The resultant clear solution was transferred into a Buchi Rotavapor and the solvent was distilled to dryness at 50° C. under a reduced pressure of about 650 mm Hg, followed by drying the solid obtained at about 45-50° C. under reduced pressure of 650 mm Hg for 90 minutes to afford 4.8 g of the title compound.

### EXAMPLE 2

Preparation of Stable Composition of Zafirlukast and Hydroxypropyl Cellulose

13 g of zafirlukast and 13 g of HPC (Klucel LF) were taken into a round bottom flask and 800 ml of dichloromethane was added. The mixture was stirred at 26° C. for 30 minutes and then filtered. The filtrate was taken into a Buchi Rotavapor flask and the solvent was distilled completely at a temperature of 50° C. and a vacuum of 0 mm Hg. The solid obtained was collected and micronized under a nitrogen pressure of 3.7 kg/cm². The micronized material was again dried at a temperature of 90° C. under a vacuum of 0 mm Hg for 12 hours to get 5.5 g of the title compound.

Residual solvent content: Dichloromethane: 91 ppm.
n-Heptane: 16 ppm.
Cyclohexane: 13 ppm.

We claim:

1. An intimate dispersion comprising zafirlukast and hydroxypropyl cellulose.

2. The intimate dispersion of claim 1, having a weight ratio of hydroxypropyl cellulose to zafirlukast about 0.1:99.9 to about 1:1.

3. The intimate dispersion of claim 1, having a weight ratio of hydroxypropyl cellulose to zafirlukast about 1:1.

4. The intimate dispersion of claim 1, wherein hydroxypropyl cellulose has an average molecular weight about 50,000 to about 1,250,000.

5. The intimate dispersion of claim 1, prepared by a process comprising removing solvent from a solution comprising zafirlukast and hydroxypropyl cellulose.

6. The intimate dispersion of claim 5, wherein a solvent comprises an alcohol, a halogenated hydrocarbon, a ketone, an ester, an ether, a hydrocarbon, a nitrile, or a mixture of any two or more thereof.

7. The intimate dispersion of claim 5, wherein a solvent comprises a halogenated hydrocarbon.

8. The intimate dispersion of claim 5, wherein a solvent comprises dichloromethane.

9. The intimate dispersion of claim 5, wherein removing solvent is conducted under a vacuum.

10. The intimate dispersion of claim 1, wherein zafirlukast is substantially amorphous.

11. A pharmaceutical composition comprising an intimate dispersion of claim 1, and at least one pharmaceutically acceptable excipient.

12. A process for preparing an intimate dispersion, comprising removing solvent from a solution comprising zafirlukast and hydroxypropyl cellulose.

13. The process of claim 12, wherein zafirlukast in an intimate dispersion is amorphous.

14. The process of claim 12, wherein a solvent comprises an alcohol, a halogenated hydrocarbon, a ketone, an ester, an ether, a hydrocarbon, a nitrile, or a mixture of any two or more thereof.

15. The process of claim 12, wherein a solvent comprises a halogenated hydrocarbon.

16. The process of claim 12, wherein a solvent comprises dichloromethane.

17. The process of claim 12, wherein solvent is removed at an elevated temperature.

18. The process of claim 12, wherein solvent is removed under a vacuum.

19. A process for preparing an intimate dispersion, comprising removing solvent at an elevated temperature and under a vacuum, from a solution comprising zafirlukast, hydroxypropyl cellulose, and a halogenated hydrocarbon.

20. The process of claim 19, wherein zafirlukast in an intimate dispersion is amorphous.
